(19) United States
(12) Patent Application Publication
(10) Pub. No.: US 2024/0091501 A1
(43) Pub. Date: Mar. 21, 2024

(54) Rapidly Inserted Central Catheter and Methods Thereof

(71) Applicant: Bard Access Systems, Inc., Salt Lake City, UT (US)

(72) Inventor: Glade H. Howell, Draper, UT (US)

(21) Appl. No.: 18/524,480

(22) Filed: Nov. 30, 2023

Related U.S. Application Data
(63) Continuation of application No. 17/006,553, filed on Aug. 28, 2020, now Pat. No. 11,890,429.
(60) Provisional application No. 62/898,408, filed on Sep. 10, 2019.

(51) Int. Cl. A61M 25/01; A61M 25/00; A61M 25/09

(52) U.S. Cl. CPC A61M 25/0105; A61M 25/0009; A61M 25/09; A61M 2025/09158

(57) ABSTRACT

Rapidly inserted central catheters (“RICC”), and methods thereof. A RICC can include a catheter tube including a first section in a distal end portion of the catheter tube, a second section in the distal end portion of the catheter tube proximal of the first section, and a junction between the first and second sections of the catheter tube. The first section of the catheter tube can be formed of a first material having a first durometer. The second section of the catheter tube can be formed of a second material having a second durometer less than the first durometer. The first and second sections of the catheter tube can have a column strength sufficient to prevent buckling of the catheter tube when inserted into an insertion site and advanced through a vasculature of a patient.

### PRIORITY

This application is a continuation of U.S. patent application Ser. No. 17/006,553, filed Aug. 28, 2020, which claims the benefit of priority to U.S. Provisional Application No. 62/898,408, filed Sep. 10, 2019, each of which is incorporated by reference in its entirety into this application.

### BACKGROUND

A central venous catheter (“CVC”) is formed of a material having a relatively low durometer, which contributes to the CVC having a lack of column strength. Due to the lack of column strength, CVCs are commonly introduced into patients and advanced through vasculatures thereof by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma. In addition, there is a relatively high potential for touch contamination due to the number of medical devices that need to be interchanged during the number of steps of the Seldinger technique. As such, there is a need to reduce the number of steps and medical devices involved in introducing a catheter into patient and advancing the catheter through a vasculature thereof.

Disclosed herein are rapidly inserted central catheters (“RICCs”) and methods thereof that address the foregoing.

### SUMMARY

Disclosed herein is a RICC including, in some embodiments, a catheter tube including a first section in a distal-end portion of the catheter tube, a second section in the distal-end portion of the catheter tube proximal of the first section, and a junction between the first and second sections of the catheter tube. The first section of the catheter tube is formed of a first material having a first durometer. The second section of the catheter tube is formed of a second material having a second durometer less than the first durometer. The first and second sections of the catheter tube have a column strength sufficient to prevent buckling of the catheter tube when inserted into an insertion site and advanced through a vasculature of a patient.

In some embodiments, the junction is a third section of the catheter tube formed of a third material having a third durometer between the first and second durometers.

In some embodiments, the junction includes a tapered distal-end portion, the first section of the catheter tube includes a flared proximal-end portion, and the tapered distal-end portion of the junction sits within the flared proximal-end portion of the first section of the catheter tube.

In some embodiments, the junction includes a proximal-end portion, the second section of the catheter tube includes a distal-end portion, and the proximal-end portion of the junction abuts the distal-end portion of the second section of the catheter tube.

In some embodiments, each section of the first and second sections of the catheter tube is welded to the junction in a heat weld.

In some embodiments, each section of the first and second sections of the catheter tube is welded to the junction in a solvent weld.

In some embodiments, the first section of the catheter tube includes a flared proximal-end portion, the second section of the catheter tube includes a tapered distal-end portion, and the tapered distal-end portion of the second section of the catheter tube sits within the flared proximal-end portion of the first section of the catheter tube.

In some embodiments, each section of the first and second sections of the catheter tube is welded to the other section in a heat weld, thereby forming the junction between the first and second sections of the catheter tube.

In some embodiments, each section of the first and second sections of the catheter tube is welded to the other section in a solvent weld, thereby forming the junction between the first and second sections of the catheter tube.

In some embodiments, the RICC includes a necked-down section about the junction. The necked-down section is configured to provide a smooth transition between the first and second sections of the catheter tube.

In some embodiments, the first section of the catheter tube is polytetrafluoroethylene, polypropylene, or polyurethane.

In some embodiments, the second section of the catheter tube is polyvinyl chloride, polyethylene, polyurethane, or silicone.

Also disclosed herein is a method of making a RICC including, in some embodiments, obtaining steps of obtaining each section of a first section, a second section, and a third section of a catheter tube. The first section of the catheter tube is formed of a first material having a first durometer. The second section of the catheter tube is formed of a second material having a second durometer. The third section of the catheter tube is formed of a third material having a third durometer between the first and second durometers. The method also includes a flaring step of flaring a proximal-end portion of the first section of the catheter tube to form a flared proximal-end portion of the first section of the catheter tube. The method also includes an inserting step of inserting a tapered distal-end portion of the third section of the catheter tube into the flared proximal-end portion of the first section of the catheter tube. The method also includes an abutting step of abutting a distal-end portion of the second section of the catheter tube and a proximal-end portion of the third section of the catheter tube. The method also includes welding steps of welding the first, second, and third sections of the catheter tube together to form a junction of the third material between the first and second sections of the catheter tube.

In some embodiments, a welding step of welding the second and third sections of the catheter tube occurs before the inserting and welding steps of inserting the tapered distal-end portion of the third section of the catheter tube into the flared proximal-end portion of the first section of the catheter tube and welding the first and third sections of the catheter tube together.

In some embodiments, each welding step of the welding steps is independently heat welding or solvent welding.

In some embodiments, the method further includes a tapering step of tapering a non-tapered distal-end portion of the third section of the catheter tube to form the tapered distal-end portion of the third section of the catheter tube.

In some embodiments, the method further includes a necking-down step of necking down the catheter tube to form a necked-down section about the junction. The necked-down section is configured to provide a smooth transition between the first, second, and third sections of the catheter tube.

Also disclosed herein is a method of making a RICC including, in some embodiments, obtaining steps of obtaining a first section of a catheter tube formed of a first material having a first durometer and obtaining a second section of the catheter tube formed of a second material having second durometer less than the first durometer. The method also includes a flaring step of flaring a proximal-end portion of the first section of the catheter tube to form a flared proximal-end portion of the first section of the catheter tube. The method also includes an inserting step of inserting a tapered distal-end portion of the second section of the catheter tube into the flared proximal-end portion of the first section of the catheter tube. The method also includes a welding step of welding the first and second sections of the catheter tube together to form a junction between the first and second sections of the catheter tube.

In some embodiments, the welding step includes heat welding or solvent welding.

In some embodiments, the method further includes a tapering step of tapering a non-tapered distal-end portion of the second section of the catheter tube to form the tapered distal-end portion of the second section of the catheter tube.

In some embodiments, the method further includes a necking-down step of necking down the catheter tube to form a necked-down section about the junction. The necked-down section is configured to provide a smooth transition between the first and second sections of the catheter tube.

Also disclosed herein is a method of a RICC including, in some embodiments, an insertion site-creating step of creating an insertion site with a needle to access a vasculature of a patient; an inserting step of inserting a distal-end portion of a catheter tube of the RICC into the insertion site over the needle; and an advancing step of advancing the distal-end portion of the catheter tube through the vasculature of the patient without use of a Seldinger technique.

In some embodiments, the method further includes a withdrawing step of withdrawing the needle from the RICC after the insertion site-creating step.

In some embodiments, the insertion site is at a right subclavian vein or a right internal jugular vein.

In some embodiments, the advancing step includes advancing the distal-end portion of the catheter tube through the right subclavian vein or the right internal jugular vein, a right brachiocephalic vein, and into a superior vena cava.

Also disclosed herein is a RICC including, in some embodiments, a catheter tube including a first section having a single lumen in a distal-end portion of the catheter tube, a second section having a pair of lumens in the distal-end portion of the catheter tube proximal of the first section, and a junction between the first and second sections of the catheter tube in which the pair of lumens transitions into the single lumen. The first section of the catheter tube is formed of a first material having a first durometer. The second section of the catheter tube is formed of a second material having a second durometer less than the first durometer. The first and second sections of the catheter tube have a column strength sufficient to prevent buckling of the catheter tube when inserted into an insertion site and advanced through a vasculature of a patient.

Disclosed herein is a RICC including, in some embodiments, a catheter tube including a first section in a distal-end portion of the catheter tube, a second section in the distal-end portion of the catheter tube proximal of the first section, and a junction between the first and second sections of the catheter tube. The first and second sections of the catheter tube are formed of a same material or different materials having substantially equal durometers provided the first and second sections of the catheter tube have a column strength sufficient to prevent buckling of the catheter tube when inserted into an insertion site and advanced through a vasculature of a patient.

These and other features of the concepts provided herein will become more apparent to those of skill in the art in view of the accompanying drawings and following description, which describe particular embodiments of such concepts in greater detail.

### BRIEF DESCRIPTION OF THE DRAWINGS

FIG. 1A illustrates a distal-end portion of a first RICC in accordance with some embodiments.

FIG. 1B illustrates a cross section of the distal-end portion of the first RICC of FIG. 1A.

FIG. 2 illustrates a cross section of a second RICC in accordance with some embodiments.

FIG. 3 illustrates a cross section of a third RICC in accordance with some embodiments.

FIG. 4A illustrates part of a method of making at least the first RICC in accordance with some embodiments.

FIG. 4B illustrates another part of the method of making at least the first RICC in accordance with some embodiments.

FIG. 5A illustrates part of a method of making at least an alternative of the first RICC in accordance with some embodiments.

FIG. 5B illustrates another part of the method of making at least the alternative of the second RICC in accordance with some embodiments.

FIG. 6A illustrates a distal-end portion of a fourth RICC in accordance with some embodiments.

FIG. 6B illustrates a cross section of the distal-end portion of the fourth RICC of FIG. 6A.

FIG. 7 illustrates another part of the method of making at least the fourth RICC in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

Unless defined otherwise, all technical and scientific terms used herein have the same meaning as commonly understood by those of ordinary skill in the art.

As set forth above, there is a need to reduce the number of steps and medical devices involved in introducing a catheter into patient and advancing the catheter through a vasculature thereof. Disclosed herein are RICCs and methods thereof that address the foregoing.

### Rapidly Inserted Central Catheters

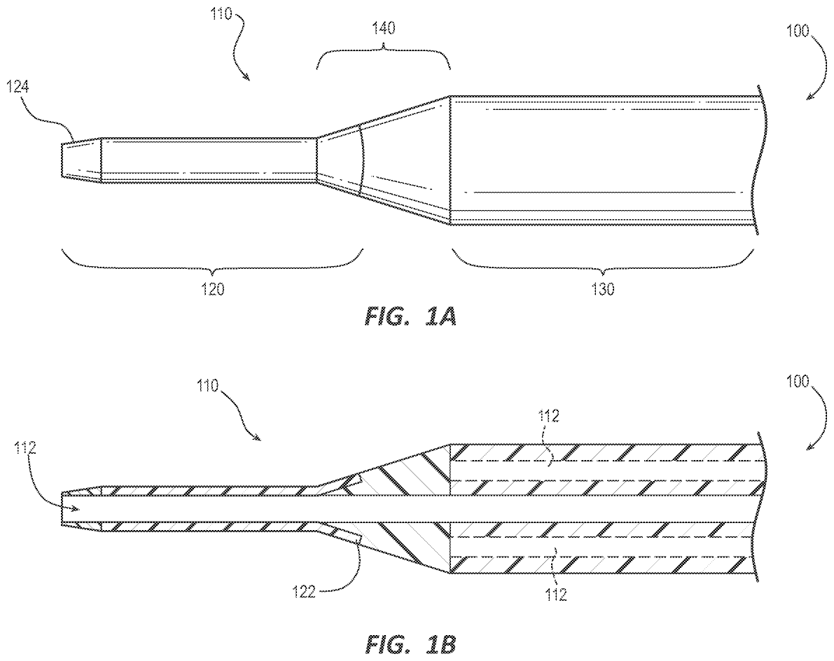

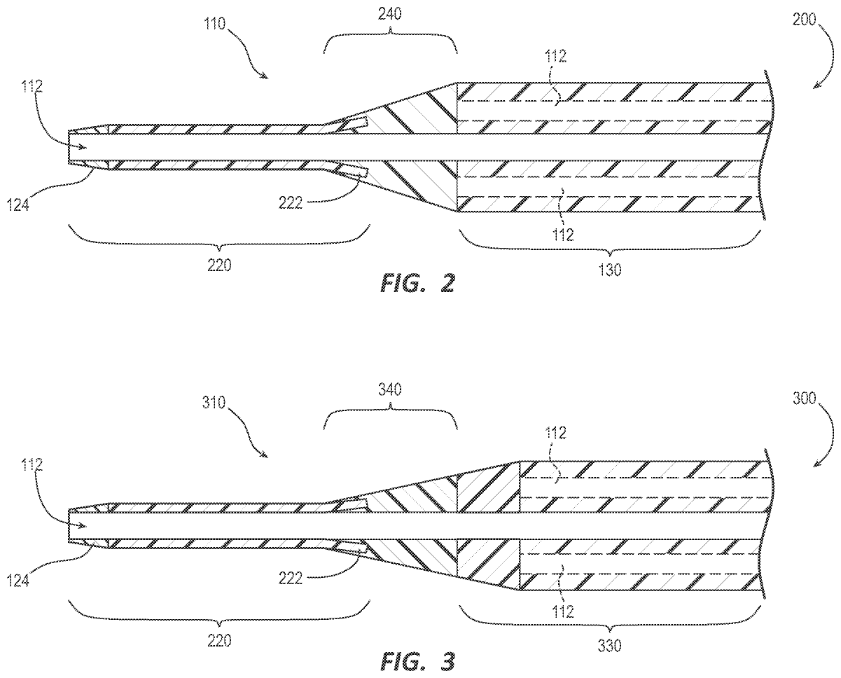

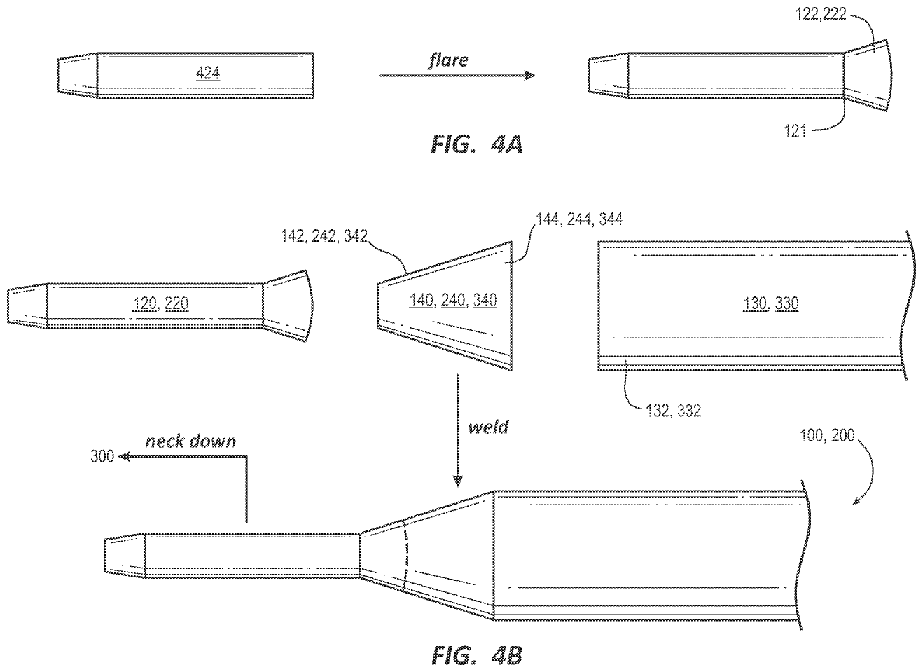

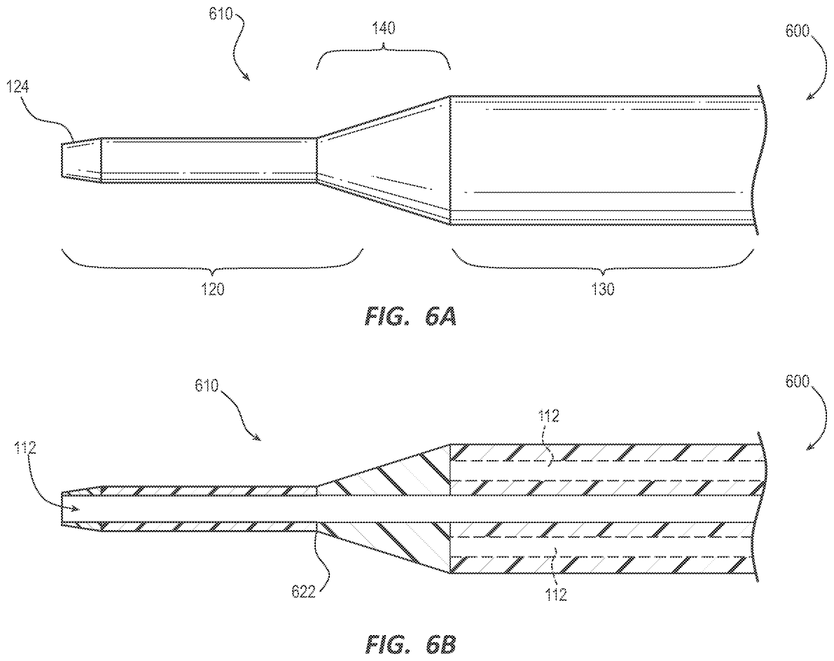

FIG. 1A illustrates a distal-end portion of a RICC 100 in accordance with some embodiments. FIG. 1B illustrates a cross section of the distal-end portion of the RICC 100 of FIG. 1A. FIG. 2 illustrates a cross section of a RICC 200 in accordance with some embodiments. FIG. 3 illustrates a cross section of a RICC 300 in accordance with some embodiments. FIG. 6A illustrates a distal-end portion of a RICC 600 in accordance with some embodiments. FIG. 6B illustrates a cross section of the distal-end portion of the RICC 600 of FIG. 6A.

As shown, the RICC 100 includes a catheter tube 110 including a first section 120 in the distal-end portion of the catheter tube 110, a second section 130 in the distal-end portion of the catheter tube 110 proximal of the first section 120, and a junction 140 between the first section 120 and second section 130 of the catheter tube 110. Likewise, the RICC 200 includes a catheter tube 210 including a first section 220 in the distal-end portion of the catheter tube 210, the second section 130 in the distal-end portion of the catheter tube 210 proximal of the first section 220, and a junction 240 between the first section 220 and second section 130 of the catheter tube 210. Further likewise, the RICC 300 includes a catheter tube 310 including the first section 220 in the distal-end portion of the catheter tube 310, a second section 330 in the distal-end portion of the catheter tube 310 proximal of the first section 220, and a junction 340 between the first section 220 and second section 330 of the catheter tube 310. Even further likewise, the RICC 600 includes a catheter tube 610 including a first section 620 in the distal-end portion of the catheter tube 610, the second section 130 in the distal-end portion of the catheter tube 610 proximal of the first section 620, and the junction 140 between the first section 620 and second section 130 of the catheter tube 610. Together, the first section 120, 220, or 620 of the catheter tube 110, 210, 310, or 610, the second section 130 or 330 of the catheter tube 110, 210, 310, or 610 and the junction 140, 240, or 340 have a column strength sufficient to prevent buckling of the catheter tube 110, 210, 310, or 610 when inserted into an insertion site and advanced through a vasculature of a patient. While the RICC 100, 200, 300, and 600 have the foregoing sections, it should be understood other sections and configurations thereof are possible.

While only a single lumen 112 is shown for the RICC 100, 200, 300, or 600, the RICC 100, 200, 300, or 600 is not limited to being a monoluminal catheter. Indeed, the RICC 100, 200, 300, or 600 can alternatively be a diluminal catheter, a triluminal catheter, a tetraluminal catheter, etc. A number of lumens 112 in one portion of the RICC 100, 200, 300, or 600 can also transition into a fewer number of lumens 112 in another portion of the RICC 100, 200, 300, or 600. For example, a pair of lumens 112 in the second section 130 of the catheter tube 110 of the RICC 100 can transition into the single lumen 112 in the first section 120 of the catheter tube 110.

The first section 120 or 220 of the catheter tube 110, 210, or 310 includes a flared proximal-end portion 122 or 222 and a tip 124 of the first section 120 or 220 of the catheter tube 110, 210, or 310 that doubles as the tip 124 of the catheter tube 110, 210, or 310. The flared proximal-end portions 122 or 222 can differ with respect to degree of flare with the flared proximal-end portion 122 of the first section 110 having a greater degree of flare than the flared proximal-end portion 222 of the first section 220 of either the second catheter tube 210 or the third catheter tube 310. While the first section 620 of the catheter tube 610 likewise includes the tip 124 that doubles as the tip 124 of the catheter tube 610, the first section 620 of the catheter tube 610 does not include a flared proximal-end portion like the flared proximal-end portion 122 or 222 of the first section 120 or 220 of the catheter tube 110, 210, or 310. Instead, a proximal-end portion 622 of the first section 620 of the catheter tube 610 continues with a same outer diameter as a distal-end portion of the first section 620 of the catheter tube 610 proximal of the tip 124.

The first section 120, 220, or 620 of the catheter tube 110, 210, 310, or 610 is formed of a first material having a first durometer. The first material can be polytetrafluoroethylene, polypropylene, or polyurethane, but the first material is not limited to the foregoing polymers. Polyurethane is advantageous in that the first section 120, 220, or 620 of the catheter tube 110, 210, 310, or 610 can be relatively rigid at room-temperature but become more flexible in vivo at body temperature, which reduces irritation to vessel walls and phlebitis.

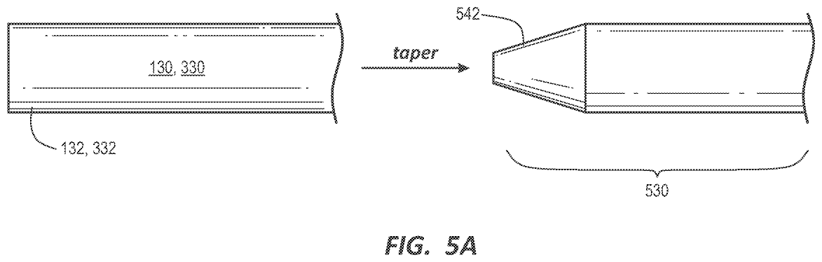

The second section 130 or 330 of the catheter tube 110, 210, 310, or 610 includes a distal-end portion 132, 232, or 332 as best seen in FIG. 4B.

The second section 130 or 330 of the catheter tube 110, 210, 310, or 610 is formed of a second material having a second durometer less than the first durometer of the first material. The first durometer and the second durometer can be on different scales (e.g., Type A or Type D), so the second durometer might not be numerically less than the first durometer. That said, the hardness of the second material can still be less than the hardness of the first material as the different scales—each of which ranges from 0 to 100—are designed for characterizing different materials in groups of the materials having a like hardness. The second material can be polyvinyl chloride, polyethylene, polyurethane, or silicone, but the first material is not limited to the foregoing polymers. Polyurethane is advantageous in that can be less thrombogenic than some other polymers.

Notwithstanding the foregoing, the first section 120, 220, or 620 and the second section 130 or 330 of the catheter tube 110, 210, 310, or 610 can be formed of a same material or different materials having substantially equal durometers provided a column strength of the catheter tube 110, 210, 310, or 610 is sufficient to prevent buckling of the catheter tube 110, 210, 310, or 610 when inserted into an insertion site and advanced through a vasculature of a patient.

The junction 140, 240, or 340 can be a third section of the catheter tube 110, 210, 310, or 610. The junction 140, 240, or 340 includes a tapered distal-end portion 142, 242, or 342 as best seen in FIG. 4B, as well as a proximal-end portion 144, 244, or 344. Each tapered distal-end portion of the tapered distal-end portions 142, 242, and 342 can differ with respect to degree of taper. For example, the tapered distal-end portion 142 of the junction 140 can have a greater degree of taper than the tapered distal-end portion 242 or 342 of the junction 240 or 340.

The junction 140, 240, or 340 is formed of a third material having a third durometer between the first durometer of the first material of the first section 120, 220, or 620 of the catheter tube 110, 210, 310, or 610 and the second durometer of the second material of the second section 130 or 330 of the catheter tube 110, 210, 310, or 610. Like that set forth above, such durometers can be on different scales (e.g., Type A or Type D), so the third durometer might not be numerically between the first durometer and the second durometer. Alternatively, the third material can have a same durometer as the first material of the first section 120, 220, or 620 of the catheter tube 110, 210, 310, or 610 or the second material of the second section 130 or 330 of the catheter tube 110, 210, 310, or 610.

The tapered distal-end portion 142, 242, or 342 of the junction 140, 240, or 340 sits at least partially within the flared proximal-end portion 122 or 222 of the first section 120 or 220 of the catheter tube 110, 210, or 310. With respect to the catheter tube 610, however, the tapered distal-end portion 142 of the junction 140 abuts the proximal-end portion 622 of the first section 620 of the catheter tube 610. The proximal-end portion 144, 244, or 344 of the junction 140, 240, or 340 abuts the distal-end portion 132 or 332 of the second section 130 or 330 of the catheter tube 110, 210, 310, or 610. Each section of the first section 120, 220, or 620 and second section 130 or 330 of the catheter tube 110, 210, 310, or 610 is independently welded to the junction 140, 240, or 340 in a heat weld or a solvent weld. If one type of welding is used, the first section 120, 220, or 620 and second section 130 or 330 of the catheter tube 110, 210, 310, or 610 can be welded to the junction 140, 240, or 340 in one heat-welding or a solvent-welding step. The flared proximal-end portion 122 of the first section 120 of the catheter tube 110 is exposed, whereas the flared proximal-end portion 222 of the first section 220 of the catheter tube 210 or 310 is concealed by an overlying portion of the junction 240 or 340 for a smoother transition. For an even smoother transition, both the second section 330 of the catheter tube 310 and the junction 340 can be necked-down in a combined necked down section about the junction 340.

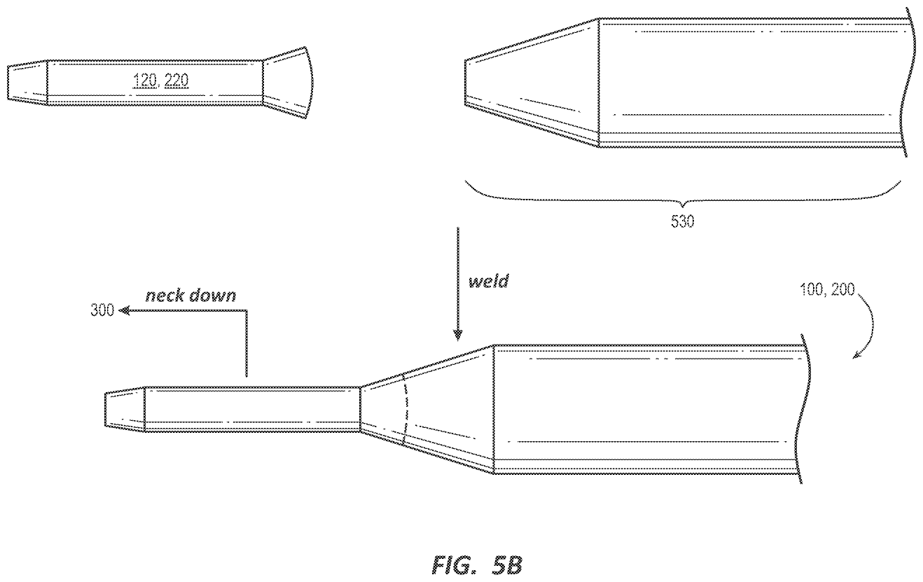

As an alternative to the foregoing, the second section 130 or 330 of the catheter tube 110, 210, 310, or 610 includes the junction 140, 240, or 340 or third section of the catheter tube 110, 210, 310, or 610. That is, the third section of the catheter tube 110, 210, 310, or 610 is not formed separately from the second section 130 or 330 of the catheter tube 110, 210, 310, or 610 and welded thereto—but integrally with the second section 130 or 330 of the catheter tube 110, 210, 310, or 610 as shown in FIG. 5A for a second section 530. Like that set forth above for the junction 140, 240, or 340, the second section 530 includes a tapered distal-end portion 542. The tapered distal-end portion 542 can differ with respect to degree of taper. For example, the tapered distal-end portion 542 of the second section 530 can have a greater degree of taper for the catheter tube 110 or 610 than for either catheter tube of the catheter tubes 210 and 310.

The tapered distal-end portion 542 of the second section 530 of the catheter tube 110, 210, or 310 sits at least partially within the flared proximal-end portion 122 or 222 of the first section 120 or 220 of the catheter tube 110, 210, or 310 or abuts the proximal-end portion 622 of the first section 620 of the catheter tube 610. The first section 120, 220, or 620 and second section 530 of the catheter tube 110, 210, 310, or 610 are welded together in a heat weld or a solvent weld. The flared proximal-end portion 122 of the first section 120 of the catheter tube 120 is exposed, whereas the flared proximal-end portion 222 of the first section 220 of the catheter tube 210 or 310 is concealed by an overlying portion of the second section 530 of the catheter tube 210 or 310 for a smoother transition. For an even smoother transition, the second section 530 of the catheter tube 310 can be necked-down in a necked down section.

### Methods

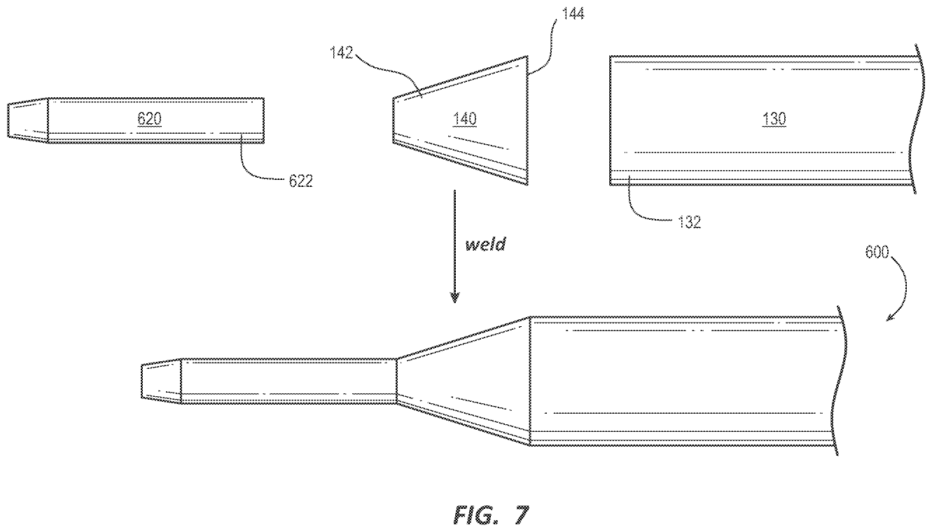

FIGS. 4A and 4B illustrate a method of making at least the RICC 100 in accordance with some embodiments. Indeed, the method shown in FIGS. 4A and 4B is also applicable to the RICC 200 and 300. FIG. 7 illustrates a method of making at least the RICC 600 accordance with some embodiments. The method of making the RICC 600 is analogous to that shown in FIG. 4B for the RICC 100 albeit without aspects of the method directed to the flared proximal-end portion 122 of the first section 120 of the catheter tube 110. For expository expediency, the method of FIGS. 4A and 4B will be primarily described with reference to making the RICC 100. Only where the method of making the RICC 200, 300, or 600 appreciably diverges from that of the RICC 100 will the method of FIGS. 4A and 4B be described with reference to the RICC 200 or 300 or the method of FIG. 7 be described with reference to the RICC 600.

The method of making the RICC 100 includes obtaining steps of obtaining each section of the first section 120, the second section 130, and the junction or third section 140 of the catheter tube 110. Again, the first section 120 of the catheter tube 110 is formed of a first material having a first durometer, the second section 130 of the catheter tube 110 is formed of a second material having a second durometer, and the third section 140 of the catheter tube 110 is formed of a third material having a third durometer between the first and second durometers. Such materials having such durometers make use of different melting temperatures and promote welds between the different sections 120, 130, and 140 of the catheter tube 110 sufficient for using the RICC 100.

The method also includes a flaring step of flaring a proximal-end portion of a nascent first section 424 of the catheter tube 110 to form the first section 120 of the catheter tube 110 with the flared proximal-end portion 122. Notably, the first section 620 of the catheter tube 610 does not include a flared proximal-end portion like the flared proximal-end portion 122 of the first section 120 of the catheter tube 110. Therefore, the method of making the RICC 600 need not include the foregoing flaring step.

The method also includes an inserting step of inserting the tapered distal-end portion 142 of the third section 140 of the catheter tube 110 into the flared proximal-end portion 122 of the first section 120 of the catheter tube 110. Notably, the first section 620 of the catheter tube 610 does not include a flared proximal-end portion like the flared proximal-end portion 122 of the first section 120 of the catheter tube 110. Therefore, the method of making the RICC 600 need not include the foregoing inserting step. Instead, the method of making the RICC 600 includes an abutting step of abutting the tapered distal-end portion 142 of the third section 140 of the catheter tube 610 into the proximal-end portion 622 of the first section 620 of the catheter tube 610.

The method also includes an abutting step of abutting the distal-end portion 132 of the second section 130 of the catheter tube 110 and the proximal-end portion 144 of the third section 140 of the catheter tube 110.

The method also includes welding steps of welding the first, second, and third sections of the catheter tube 110 together to form the junction 140 of the third material between the first section 120 and second section 130 of the catheter tube 110.

The welding step of welding the second section 130 and third section 140 of the catheter tube occurs before the inserting and welding steps of inserting the tapered distal-end portion 142 of the third section 140 of the catheter tube 110 into the flared proximal-end portion 122 of the first section 120 of the catheter tube 110 and welding the first section 120 and third section 140 of the catheter tube 110 together. With respect to the method of making the RICC 600, the welding step of welding the second section 130 and third section 140 of the catheter tube occurs before the abutting and welding steps of abutting the tapered distal-end portion 142 of the third section 140 of the catheter tube 610 and the proximal-end portion 622 of the first section 620 of the catheter tube 610 and welding the first section 620 and third section 140 of the catheter tube 610 together. Each welding step of the welding steps is independently heat welding (e.g., radiofrequency [“RF” ] welding) or solvent welding.

Flaring the proximal-end portion of the nascent first section 424 of the catheter tube 110 to form the flared proximal-end portion 122 of the first section 120 of the catheter tube 110 and inserting the tapered distal-end portion 142 of the third section 140 of the catheter tube 110 into the flared proximal-end portion 122 of the first section 120 of the catheter tube 110 makes it possible to place an RF-welding element a short distance from a start of the flared proximal-end portion 122 of the first section 120 of the catheter tube 110. This reduces a risk of causing a defect or a weak spot in the catheter tube 110. The RF-welding element would touch off a short distance distal of portion 121 and a short distance proximal of the portion 121 where the flared proximal-end portion 122 of the first section 120 of the catheter tube 110 begins so as to avoid too much heat exposure to the thin wall of the first section 120 of the catheter tube 110.

The method further includes a tapering step of tapering a non-tapered distal-end portion of the third section 140 of the catheter tube 110 to form the tapered distal-end portion 142 of the third section 140 of the catheter tube 110.

For at least the RICC 300, the method further includes a necking-down step of necking down the catheter tube 310 to form a necked-down section about the junction 340. The necked-down section is configured to provide a smooth transition between the first, second, and third sections of the catheter tube 310.

FIGS. 5A and 5B illustrate a method of making the alternative of the RICC 100 in accordance with some embodiments.

The method of making the alternative to the RICC 100 includes obtaining steps of obtaining the first section 120 of the catheter tube 110 formed of a first material having a first durometer and the second section 530 of the catheter tube 110 formed of a second material having second durometer less than the first durometer. The method also includes a flaring step of flaring the nascent first section 424 of the catheter tube 110 as shown in FIG. 4B to form the flared proximal-end portion 122 of the first section 120 of the catheter tube 110. The method also includes an inserting step of inserting the tapered distal-end portion 542 of the second section 530 of the catheter tube 110 into the flared proximal-end portion 122 of the first section 120 of the catheter tube 110. The method also includes a welding step of heat or solvent welding the first section 120 and second section 530 of the catheter tube 110 together to form a junction therebetween.

The method further includes a tapering step of tapering a non-tapered distal-end portion 132 of the second section 130 of the catheter tube 110 to form the tapered distal-end portion 542 of the second section 530 of the catheter tube 110. Forming the tapered distal-end portion 542 of the second section 530 of the catheter tube 110 can be accomplished by removing some material from the non-tapered distal-end portion 132 of the second section 130 of the catheter tube 110 or melting or dissolving some of the material of the non-tapered distal-end portion 132 of the second section 130 of the catheter tube 110 during the welding step.

For at least the RICC 300, the method further includes a necking-down step of necking down the catheter tube 310 to form a necked-down section about the junction between the first section 120 and second section 530 of the catheter tube 110. The necked-down section is configured to provide a smooth transition between the first section 220 and second section 530 of the catheter tube 310.

The method of making each RICC of the RICCs 100, 200, and 300 further includes a mandrel-mounting step of mounting the sections used in making the RICCs 100, 200, and 300 on mandrels to keep lumens thereof from collapsing during the more welding steps.

A method of the RICC 100 includes an insertion site-creating step of creating an insertion site with a needle to access a vasculature of a patient; an inserting step of inserting the distal-end portion of the catheter tube 110 of the RICC 100 into the insertion site over the needle; and an advancing step of advancing the distal-end portion of the catheter tube 110 through the vasculature of the patient without use of a Seldinger technique. A smaller outer diameter of the first section 120 of the RICC 100 facilitates the inserting step, which requires pushing through at least skin tissue. In addition, the transition between the first section 120 and second section 130 eliminates snagging on at least the skin tissue making it possible to insert the RICC 100 like a peripheral intravenous (“IV”) catheter.

The method further includes a withdrawing step of withdrawing the needle from the RICC 100 after the insertion site-creating step.

The insertion site is at a right subclavian vein or a right internal jugular vein.

The advancing step includes advancing the distal-end portion of the catheter tube 110 through the right subclavian vein or the right internal jugular vein, a right brachiocephalic vein, and into a superior vena cava.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A rapidly inserted central catheter (“RICC”), comprising:
a first section formed of a first material having a first durometer, the first section including a flared proximal end portion;
a second section formed of a second material having a second durometer less than the first durometer, the second section proximal of the first section; and
a third section positioned between the first section and the second section, the third section formed of a third material having a third durometer less than the first durometer but greater than the second durometer, the third section including a tapered distal end portion configured for positioning within the flared proximal end portion of the first section.

2. The RICC according to claim 1, wherein the first section, the second section, and the third section are in a distal portion of the RICC.

3. The RICC according to claim 2, wherein the first section and the second section possess a column strength sufficient to prevent buckling when the RICC is inserted into and moved through a vasculature of a patient.

4. The RICC according to claim 2, wherein the third section includes a proximal end portion, wherein the second section includes a distal end portion, and wherein the proximal end portion of the third section abuts the distal end portion of the second section.

5. The RICC according to claim 1, wherein each of the first section and the second is welded to the third section in a heat weld.

6. The RICC according to claim 1, wherein each of the first section and the second is welded to the third section in a solvent weld.

7. The RICC according to claim 1, wherein the third section includes a necked-down section configured to provide a smooth transition between the first section and the second section.

8. The RICC according to claim 1, wherein the first material is selected from the group consisting of polytetrafluoroethylene, polypropylene, and polyurethane.

9. The RICC according to claim 1, wherein the second material is selected from the group consisting of polyvinyl chloride, polyethylene, polyurethane, and silicone.

10. The RICC according to claim 1, wherein the first section has a single lumen, and wherein the second section has a pair of lumens that transition into the single lumen in the third section.

11. A rapidly inserted central catheter (“RICC”), comprising:
a first section formed of a first material having a first durometer, the first section including a flared proximal end portion; and
a second section formed of a second material having a second durometer less than the first durometer, the second section including a tapered distal end portion positioned within the flared proximal end portion of the first section to form a junction between the first section and the second section.

12. The RICC according to claim 11, wherein the first section and the second section are in a distal portion of the RICC.

13. The RICC according to claim 12, wherein the first section and the second section possess a column strength sufficient to prevent buckling when the RICC is inserted into and moved through a vasculature of a patient.

14. The RICC according to claim 11, wherein the first section is welded to the second at the junction in a heat weld.

15. The RICC according to claim 11, wherein the first section is welded to the second at the junction in a solvent weld.

16. The RICC according to claim 11, wherein the RICC includes a necked-down section at the junction configured to provide a smooth transition between the first section and the second section.

17. The RICC according to claim 11, wherein the first material is selected from the group consisting of polytetrafluoroethylene, polypropylene, and polyurethane.

18. The RICC according to claim 11, wherein the second material is selected from the group consisting of polyvinyl chloride, polyethylene, polyurethane, and silicone.

19. The RICC according to claim 11, wherein the first section has a single lumen, and wherein the second section has a pair of lumens that transition into the single lumen in the junction.
